(19) United States
(12) Patent Application Publication
(10) Pub. No.: US 2009/0312367 A1
(43) Pub. Date: Dec. 17, 2009

(54) COMBINATION OF MODAFINIL AND AN ANTAGONIST OR INVERSE AGONIST OF THE H3 RECEPTOR

(75) Inventors: Jean-Charles Schwartz, Paris (FR); Jeanne-Marie Lecomte, Paris (FR)

Correspondence Address: OCCHIUTI ROHLICEK & TSAO, LLP, 10 FAWCETT STREET, CAMBRIDGE, MA, 02138, US

(73) Assignee: Bioprojet, Paris (FR)

(21) Appl. No.: 12/374,517

(22) PCT Filed: Jul. 20, 2007
(86) PCT No.: PCT/FR07/51702

§ 371 Date: Jan. 21, 2009

(30) Foreign Application Priority Data
Jul. 21, 2006 (FR) 0606697

(51) Int. Cl. A61K 31/445; A61K 31/165

(52) U.S. Cl. 514/317; 514/618

(57) ABSTRACT

The invention relates to a combination of modafinil and at least one histamine H3 receptor antagonist or inverse agonist, which can be used in particular for the treatment of narcolepsy-cataplexy and more generally for disorders of sleep, wakefulness and vigilance.

The invention relates to a combination of modafinil and a histamine H3 receptor antagonist or inverse agonist, in particular for the treatment of narcolepsy-cataplexy.

Narcolepsy-cataplexy, or Gelineau syndrome, is a rare but serious disorder characterized by excessive daytime sleepiness which can be an extreme hindrance to normal professional and social activities, and which is accompanied by more or less frequent attacks of cataplexy (a sudden loss of muscle tone triggered by emotions as varied as laughter or fear) and erratic episodes of REM sleep (during wakefulness and during sleep), sometimes associated with hypnagogic hallucinations. Moreover, individuals with narcolepsy have various degrees of cognitive impairment and tend to be obese (reviewed by Dauvilliers et al., Clin. Neurophysiol., 2003, 114, 2000; Baumann and Bassetti, Sleep Med. Rev., 2005, 9, 253).

The disorder is caused by the loss of a group of neurons in the brain which produce two peptides, orexins, also known as hypocretins, located in the anterior hypothalamus and projecting to the main groups of aminergic neurons which regulate wakefulness and sleep. Patients with the disorder generally have very low levels of orexins in cerebrospinal fluid. Orexin knock-out mice display many of the symptoms seen in narcoleptic subjects, confirming the role of these peptides and thereby providing an excellent animal model of the disease (Chemelli et al., Cell, 1999, 98, 437).

Several types of treatments which can improve the symptoms of narcolepsy already exist, although they do not completely relieve symptoms and, furthermore, can cause significant side effects limiting their usefulness.

For instance, amphetamines or analogues such as methylphenidate which release catecholamines are used to treated daytime sleepiness, but these agents induce a state of excessive excitation as well as cardiovascular disturbances and also carry a potential for drug addiction.

Modafinil, a drug whose mechanism of action is unclear, also improves daytime sleepiness without causing as many side effects as amphetamines. Nonetheless, its efficacy is limited and it can cause headaches and nausea, particularly at high doses. Moreover amphetamines and/or modafinil do not appear to improve some of the most disabling symptoms of the disease, particularly cataplexy attacks, cognitive deficits and weight gain. With regard to cataplexy, treatments include antidepressants and oxybate. Effectiveness of the former has not been demonstrated (Cochrane Database Syst. Rev., 2005, 20, 3), and the latter is a drug of illegal abuse and its use is restricted.

It has also been shown that histamine H3 receptor antagonists induce the activation of histaminergic neurons in the brain which release histamine, a neurotransmitter with a crucial role in maintaining wakefulness (Schwartz et al., Physiol. Rev. 1991, 71, 1).

### SUMMARY OF THE INVENTION

In an unexpected manner, the inventors have shown that histamine H3 receptor antagonists or inverse agonists exhibit an anticataplectic potential. This is strongly potentiated by modafinil which, nevertheless, does not by itself exert any anticataplectic activity, even at high doses.

On the basis of these findings, acquired through the use of a reliable orexin knock-out mouse model, the inventors propose a complete treatment for the entire spectrum of narcoleptic symptoms, by combining an H3 receptor antagonist or inverse agonist, and modafinil.

A subject of the invention is therefore a pharmaceutical composition comprising, in a physiologically acceptable medium, modafinil and at least one histamine H3 receptor antagonist or inverse agonist.

Another subject of the invention is the use of modafinil for preparing a medicament for treating a disorder of sleep, wakefulness and vigilance, in combination with at least one histamine H3 receptor antagonist or inverse agonist. Said combination is particularly useful for treating narcolepsy-cataplexy, and for preventing cataplexy attacks.

According to a particular embodiment, modafinil and the H3 receptor antagonist or inverse agonist can be combined within the same pharmaceutical composition. They can also be intended for separate administration. In this regard, the invention additionally provides for a kit comprising, within a same package,
a pharmaceutical composition A comprising modafinil in a physiologically acceptable medium;
a pharmaceutical composition B comprising a histamine H3 receptor antagonist or inverse agonist, in a physiologically acceptable medium.

### DETAILED DESCRIPTION OF THE INVENTION

### Modafinil

The present invention makes use of modafinil which may be in its racemic form or as one or the other of its optical isomers.

U.S. Pat. No. 4,177,290 describes modafinil, also called 2-benzhydrylsulfinylthanamide, in racemic form.

In the present invention, however, the levorotatory enantiomer is preferred, as described in U.S. Pat. No. 4,927,855.

The invention also comprises the hydrates and solvates of modafinil.

### H3 Receptor Antagonists or Inverse Agonists

The invention makes use of histamine H3 receptor antagonists or inverse agonists. The term “inverse agonist” refers to the property of H3 receptor ligands to reverse the constitutive activity of the receptor. As a general rule an inverse agonist exerts the opposite effect of an agonist and both of these effects are blocked by an antagonist. An imidazole derivative, in particular, can be used as histamine H3 receptor antagonist or inverse agonist. However, the antagonist or inverse agonist compounds described in patent application WO00/06254 are preferably used.

Thus, in a preferred embodiment, the histamine H3 receptor antagonist or inverse agonist is a compound represented by formula (I)

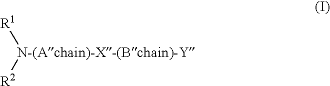

where:
R¹ and R² are the same or different, each independently representing:
   an alkyl or cycloalkyl,
      or taken together with the nitrogen atom to which they are bound,
   a saturated nitrogen ring

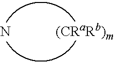

with m from 2 to 8, or
a non-aromatic unsaturated nitrogen ring

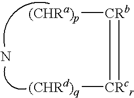

with p and q independently ranging from 0 to 3 and r from 0 to 4, provided that p and q are not simultaneously 0 and that 2≦p+q+r≦8,

R^{a-d} independently being a hydrogen atom or an alkyl, cycloalkyl or carboalkoxy group, or
a morpholino group or
an N-substituted piperazino group

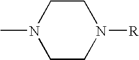

R being an alkyl, cycloalkyl, carboalkoxy, aryl, arylalkyl, alkanoyl or aroyl group.
i) the A″ chain is selected in the group consisting of linear or branched, saturated or unsaturated hydrocarbon chains, containing 1 to 6 carbon atoms, the hydrocarbon chain optionally being interrupted by a heteroatom which can be a sulfur atom,
ii) X″ is selected in the group consisting of oxygen and sulfur atoms, —NH—, —NHCO—, —N(alkyl)CO—, —NHCONH, —NH—CS—NH—, —NHCS—, —O—CO—, —CO—O—, —OCONH—, —OCON(alkyl)-, —OCON(alkene), —OCONH—CO—, —CONH—, —CON(alkyl)-, —SO—, —CO—, —CHOH—, —N(alkyl saturated or insaturated), —S—C(═NY″)—N—Y″— with Y″ the same or different and —NR_{″}C(═NR″_{″})—NR′_{″}, where R_{″} and R′_{″} represent a hydrogen atom or an alkyl moiety and R″_{″} represents a hydrogen atom or another electronegative group which can be selected from among a cyano or COY₁″ group, Y₁″ representing an alkoxy group;
iii) The B″ chain is selected in the group consisting of an aryl, arylalkyl, arylalkanoyl group; a linear alkyl chain —(CH₂)ₙ—, n ranging from 1 to 5, or a branched alkyl chain containing 2 to 8 carbon atoms, the alkyl chain optionally being interrupted by one or more oxygen or sulfur atoms; and a —(CH₂)_{n″}—O— or —(CH₂)_{n″}—S— group where n is equal to 1 or 2; and
iv) Y″ is selected in the group consisting of a linear or branched alkyl group containing 1 to 8 carbon atoms; a cycloalkyl group containing 3 to 6 carbon atoms; a bicycloalkyl group; a cycloalkenyl group; an aryl group optionally substituted with a phenyl group; a heterocyclic moiety with 5 or 6 elements containing one or two heteroatoms selected from among nitrogen and sulfur, the heterocyclic moiety optionally being substituted; and a bicyclic moiety resulting from the fusion of a benzene nucleus to a heterocycle such as defined hereinabove;
   Or
i′) the A″ chain is selected in the group consisting of a linear or branched, saturated or unsaturated —(CH₂)_{n″}— alkyl group where n″ is a whole number from 1 to 8; a linear or branched alkene group comprising from 1 to 8 carbon atoms; and a linear or branched alkyne group comprising from 1 to 4 carbon atoms;
ii′) the group X″ is selected from among —OCONH—, OCON(alkyl)-, —OCON(alkene)-, —OCO—, —OCOSNH—, —CH₂—, —O—, —OCH₂CO—, —S—, —CO—, —CS—, an amine or a saturated or unsaturated alkyl group;
iii′) the B″ chain is selected from among alkyl groups comprising from 1 to 8 carbon atoms; and —(CH₂)_{n″}(heteroatom)— where the heteroatom is preferably an oxygen or sulfur atom; n″ being a whole number from 1 to 5; and
iv′) the group Y″ represents a phenyl group which is unsubstituted, or mono- or polysubstituted with one or more substituents which are the same or different selected from among the halogen atoms, OCF₃, CHO, CF₃, SO₂N(alkyl)₂ such as SO₂N(CH₃)₂, NO₂, S(aryl), SCH₂(phenyl), a linear or branched alkene, a linear or branched alkyne optionally substituted with a trialkyl silyl moiety, —O(alkyl)-, —O(aryl), —CH₂CN, a ketone, an aldehyde, a sulfone, an acetal, an alcohol, an alkyl, —CH═CH—CHO, —C(alkyl)═N—OH, —C(alkyl)═N—O(alkyl) and other ketone derivatives, —CH═NOH, —CH═NO(alkyl) and other aldehyde derivatives, —C(alkyl)═NH—CONH2, and O-phenyl or the group —OCH2(phenyl), —C(cycloalkyl)═NOH, —C(cycloalkyl)═N—O(alkyl); a heterocycle optionally substituted, a cycloalkyl; a bicyclic group and preferably a norbomyl group; a phenyl nucleus fused to a heterocycle comprising a nitrogen heteroatom or to a carbocycle or to a heterocycle containing a ketone function; a linear or branched alkyl group comprising from 1 to 8 carbon atoms; a linear or branched alkyne group comprising from 1 to 8 carbon atoms and in particular 1 to 5 carbon atoms; a linear or branched alkyl group mono- or polysubstituted with phenyl groups which are unsubstituted or mono- or polysubstituted; a phenylalkyl ketone in which the alkyl group is linear or branched or cyclic; a substituted or unsubstituted benzophenone; a linear, branched or cyclic, substituted or unsubstituted phenyl alcohol; a linear or branched alkene; a piperidyl group; a cycloalkyl phenyl group; a polycyclic group, in particular a fluorenyl group, a naphthyl or polyhydronaphthyl group or an indanyl group; a phenol group; a ketone or ketone derivative; a diphenyl group, a phenoxyphenyl group; a benzyloxyphenyl group. In an especially preferred manner the Y″ group is a halogen.

Unless expressly indicated otherwise, the term “alkyl” designates a group comprising from 1 to 8 carbon atoms, preferably from 1 to 6 carbon atoms, and the terms “alkene” and “alkyne” designate groups comprising from 2 to 8 carbon atoms, preferably from 2 to 6 carbon atoms.

The compound may also exist in the form of its pharmaceutically acceptable salts or hydrates or hydrated salts or polymorphic crystalline structures or optical isomers, racemates, diasteromers or enantiomers, exhibiting the function of an antagonist or inverse agonist ligand of histamine H3 receptors.

Preferred compounds are compounds represented by formula (I), where:
—NR¹R² represents a piperidyl group unsubstituted or substituted with one or more alkyl groups, preferably methyl groups;
the A″ chain is a —(CH₂)ₓ— chain with x being a whole number from 1 to 6, preferably from 1 to 4, more preferably x=3;
X″ is an oxygen atom;
the B″ chain is a —(CH₂)_{y}— group with y being a whole number from 1 to 4, preferably y=2 or y=3;
Y″ is a phenyl group unsubstituted or substituted with one or more halogen atoms, or with one or more alkyl groups.

Preferably, NR¹R² represents an unsubstituted piperidyl group, and Y″ is a phenyl group substituted with a halogen atom, preferably chlorine.

A particularly preferred compound is:

3-(4-chlorophenyl)propyl-3-piperidinopropyl ether, also called 1-{3-[3-(4-chlorophenyl)propoxy]propyl}piperidine.

Other inverse agonists of the H3 receptor are described in the following documents: EP 197 840; EP 494 010; WO93/14070; WO96/29315; WO92/15567; WO93/20061; WO93/20062; WO95/11894; U.S. Pat. No. 5,486,526; WO93/12107; WO93/12108; WO95/14007; WO95/06037; WO97/29092; EP 680960; WO96/38141; WO96/38142; WO96/40126; Plazzi et al., Eur. J. Med. Chem. 1995, 30, 881; Clitherow et al., Bioorg. & Med. Chem. Lett. 6 (7), 883-838 (1996); Wolin et al., Bioorg. & Med. Chem. Left; 8, 2157 (1998); as well as WO 03/11856; WO03/24928; WO03/24929; WO 02/79168; WO02/24695; WO02/12224; WO02/32893; WO02/12190; US 2002183309; WO02/76925; WO02/13821; US 2002111340; WO02/06223; WO01/81317; WO1/74810; WO01/74813; WO01/68652; WO01/68651; WO01/74815; WO01/74814; WO01/66534; U.S. Pat. No. 6,166,060; U.S. Pat. No. 6,100,279; U.S. Pat. No. 6,034,251; EP978512; WO00/06254; WO00/42023; WO00/53596; WO00/23438; WO00/06552; WO00/64884; WO00/63208; U.S. Pat. No. 5,932,596; WO99/05114; U.S. Pat. No. 6,008,240; WO99/24421; WO99/42458; WO 99/05141; U.S. Pat. No. 5,990,317; WO99/05115; U.S. Pat. No. 5,869,479; U.S. Pat. No. 5,837,718; U.S. Pat. No. 5,639,775; U.S. Pat. No. 5,463,074; WO93/12093; U.S. Pat. No. 5,217,986; WO2006046131; WO2006035308; WO2006024955; WO2006019833; WO2006018260; WO2006011043; WO2006011042; US 2006019998; US 2006014733; WO2006004937; WO2006000914; WO2005123723; WO2005123716; U.S. Pat. No. 2005282811; WO2005117865; US 2005245543; WO2005113536; WO2005113551; WO2005111036; WO2005105744; WO2005096734; WO2005097751; WO2005097740; WO2005097778; WO2005089761; WO2005082893; EP1571145; WO2005080361; WO2005077953; WO2005077905; EP1556046; EP1554243; WO2005058837; WO2005040144; WO2005037810; WO2005028438; WO2005014571; WO2005014579; U.S. Pat. No. 6,855,560; WO2005009976; WO2005009471; WO2005007644; WO2005000315; WO2005000217; US 2004260100; US 2004248899; WO2004101559; WO2004101546; US 20040224952; US 2004220225; WO2004089373; WO2004089410; WO2004087938; EP1474132; US 2004198743; EP1463817; WO2004076388; EP1451225; WO2004069338; US 2004156845; WO2004066960; EP1444340; US 2004152704; US 2004147577; US 2004138234; WO2004056369; US 2004127718; WO2004054973; EP1428820; US 2004110748; US 2004110746; WO2004043458; US 2004097483; WO2004037788; WO2004037257; WO2004035556; WO2004026837; WO2004024707; US 2004048843; WO2004018432; WO2003011856; US 2004029943; US 2004019039; US 2004019099; US 2004006120; U.S. Pat. No. 6,673,829; WO02072570; US 2004002604; WO2004000831; US 2003236259; WO03104235; WO03103669; WO03088967; US 2003191112; US 2003186963; WO03070722; WO03066604; WO03064411; US 2003135056; US 2003113309; WO03044059; WO03042359; WO03040106; WO03039245; WO03031432; US 2003069295; EP1277477; WO03004480; WO03004637; WO0174773; US 2002198237; U.S. Pat. No. 6,489,337; US 2002151565; WO02072093; US 2002132755; U.S. Pat. No. 6,448,282; US 2002103235; U.S. Pat. No. 6,417,218; US 2002086859; US 2002082278; US 2002082272; WO0244141; WO0240461; US 2002058659; US 2002042400; WO0224659; WO0224658; WO0224657; US 2002035103; WO0215905; WO2002016340; WO2002012214; US 2001049385; US 2001049367; WO0168816; WO0168703; WO0168665; WO0146414; WO0130346; U.S. Pat. No. 6,136,559; WO0020011; U.S. Pat. No. 5,990,147; WO9924406; WO9924405; WO9806394; U.S. Pat. No. 5,708,171; U.S. Pat. No. 5,633,382; WO9640126; WO9638142; WO9638141; WO9629315; WO9314070; U.S. Pat. No. 5,486,526; WO9511894; WO9506037; EP0618905; WO9320062; WO9320061; WO9301812; WO9215567.

In particular, the following individual compounds may be mentioned:
3-phenylpropyl 3-piperidinopropyl ether;
1-[5-(4-acetamidophenoxy)-pentyl]-pyrrolidine;
1-(3-[(4-oxobutyl)phenoxyl]propyl)piperidine;
1-(3-[4-(1-hydroxypropyl)phenoxy]propyl)-3-methylpiperidine;
1-(3-[4-(1-hydroxypropyl)phenoxy]propyl)-4-methylpiperidine;
1-[3-(4-cyanophenoxy)-propyl]-piperidine;
N-[3-(4-cyanophenoxy)-propyl]-hexamethylneimine;
1-[3-(4-acetylphenoxy)-propyl]-3-methylpiperidine;
1-(3-[4-(1-ethoxypropyl)phenoxy]propyl)-2-methylpiperidine oxime;
1-[3-(4-bromophenoxy)propyl]piperidine;
1-[3-(4-isopropylphenoxy)propyl]piperidine;
1-[3-(4-sec-butylphenoxy)propyl]piperidine;
1-[3-(4-propylphenoxy)propyl]piperidine;
1-[3-(4-ethylphenoxy)propyl]piperidine.

Said compounds are described in application WO00/06254.

The following compounds may also be mentioned:
1-{3-[3-(3,4-dimethoxyphenyl)propoxy]propyl}pyrrolidine;
trans-1-{3-[3-(3,4-dimethoxyphenyl)allyloxy]propyl}piperidine;
1-{3-[3-(3,4-dimethoxyphenyl)propoxy]propyl}piperidine;
1-{3-[3-(4-methylphenyl)propoxy]propyl}piperidine;
1-{3-[3-(2-naphthyl)propoxy]propyl}piperidine;
1-{3-[3-(4-hydroxy-3-methoxyphenyl)propoxy]propyl}piperidine;
1-{3-[3-(4-fluorophenyl)propoxy]propyl}pyrrolidine;
trans-1-{3-[3-(4-fluoro-3-methoxyphenyl)allyloxy]propyl}pyrrolidine;
1-{3-[3-(4-fluoro-3-methoxyphenyl)propoxy]propyl}pyrrolidine;
1-{3-[3-(4-fluoro-3-methylphenyl)propoxy]propyl}pyrrolidine;
1-{3-[3-(4-fluoro-2-methoxyphenyl)propoxy]propyl}pyrrolidine;
trans-1-{3-[3-(benzofuran-5-yl)allyloxy]propyl}pyrrolidine;
1-{3-[3-(2,3-dihydrobenzo[1,4]dioxin-6-yl)allyl]oxy}propylpiperidine;
trans-1-{3-[3-(benzodioxol-5-yl)allyloxy]propyl}pyrrolidine;
trans-1-{3-[4-(N,N-dimethylcarbamoyl)phenoxy]propyl}-3,5-dimethylpiperidine;
trans-1-{3-[4-(N,N-tetramethynecarbamoyl)phenoxy]propyl}-3,5-dimethylpiperidine;
1-[3-(4-benzoylphenyl)propoxy]piperidine;
1-[3-(4-cyanomethylphenyl)propoxy]piperidine;
trans-1-{3-[4-(1-hydroxy-1-methylthyl)phenoxy]propyl}-3,5-dimethylpiperidine;
(RS)-1-{3-[4-(1-hydroxy-1-methylthyl)phenoxy]propyl}-3-methylpiperidine;
1-{3-[4-(1-hydroxy-1-propylbutyl)phenoxy]propyl}piperidine;
1-{3-[4-(1-hydroxycyclopentyl)phenoxy]propyl}piperidine
1-{3-[4-(1-hydroxy-1-allylbut-3-enyl)phenoxy]propyl}piperidine;
trans-1-[3-(4-isopropenylphenoxy)propyl]-3,5-dimethylpiperidine;
trans-1-[3-(4-styrylphenoxy)propyl]piperidine;
(3S,5S)-1-{3-[4-(trans-4-dimethylaminocyclohex-1-yl)phenoxy]propyl}-3,5-dimethylpiperidine;
1-{3-[4-(benzyloxy)phenoxy]propyl}piperidine;
trans-3,5-dimethyl-1-[3-(4-phenoxyphenoxy)propyl]piperidine;
6-[4-(3-piperidinopropoxy)phenyl]-2,3,4,5-tetrahydropyridine;
trans-6-{4-[3-(3,5-dimethylpiperidino)propoxy]phenyl}-2,3,4,5-tetrahydro-pyridine;
trans-1-{3-[4-(4,5-dihydro-3H-pyrrol-2-yl)phenoxy]propyl}-3,5-dimethylpiperidine;
1-{3-[4-(cis-4-dimethylaminocyclohex-1-yl)phenoxy]propyl}piperidine;
1-{3-[4-(trans-4-dimethylaminocyclohex-1-yl)phenoxy]propyl}piperidine;
1-{3-[4-(cis-4-tetramethylnaminocyclohex-1-yl)phenoxy]propyl}piperidine;
1-{3-[4-(trans-4-tetramethylnaminocyclohex-1-yl)phenoxy]-propyl}piperidine;
trans-1-{3-[4-(cis-4-dimethylaminocyclohex-1-yl)phenoxy]propyl}-3,5-dimethylpiperidine;
trans-1-{3-[4-(trans-4-dimethylaminocyclohex-1-yl)phenoxy]propyl}-3,5-dimethylpiperidine;
1-{3-[(biphenyl-4-yl)oxy]propyl}pyrrolidine;
trans-1-{3-[(biphenyl-4-yl)oxy]propyl}-3,5-dimethylpiperidine;
(3S,5S)-1-{3-[(biphenyl-4-yl)oxy]propyl}-3,5-dimethylpiperidine;
1-{3-[(4′-methylbiphenyl-4-yl)oxy]propyl}piperidine;
1-{3-[(4′-methoxybiphenyl-4-yl)oxy]propyl}piperidine;
(RS)-1-{3-[(biphenyl-4-yl)oxy]propyl}-3-methylpiperidine;
trans-3,5-dimethyl-1-{3-[(4′-methylbiphenyl-4-yl)oxy]propyl}piperidine;
1-{3-[(2′-methylbiphenyl-4-yl)oxy]propyl}piperidine;
1-{3-[4-(3-thienyl)phenoxy]propyl}piperidine;
1-{[3-{4-(4-pyridyl)phenoxy]propyl}piperidine;
trans-3,5-dimethyl-1-{3-[4-(4-pyridyl)phenoxy]propyl}piperidine;
1-{3-[4-(3-pyridyl)phenoxy]propyl}piperidine;
trans-3,5-dimethyl-1-{3-[4-(pyrrol-1-yl)phenoxy]propyl}piperidine;
trans-3,5-dimethyl-1-{3-[4-(pyrazol-3-yl)phenoxy]propyl}piperidine; di-1,1′-{(biphenyl-4,4′-diyl)bis[oxy(propan-1,3-diyl)]}piperidine;
4-(3-{[4′-(3-piperidinopropoxy)biphenyl-4-yl]oxy}propyl)morpholine;
1-(3-{[4′-(3-piperidinopropoxy)biphenyl-4-yl]oxy}propyl)pyrrolidine;
di-1,1′-{(biphenyl-4,4′-diyl)bis[oxy(propan-1,3-diyl)]}pyrrolidine;
di-1,1′-{methylnebis[(phenyl-1,4-diyl)oxy(propan-1,3-diyl)]}piperidine;
(3S,5S)-1-{3-[4-(trans-4-dimethylaminocyclohex-1-yl)phenoxy]propyl}-3,5-dimethylpiperidine;
(3S)-1-{3-[4-(trans-4-dimethylaminocyclohex-1-yl)phenoxy]propyl}-3-methylpiperidine;
(3S)-3-methyl-1-{3-[4-(4-pyridyl)phenoxy]propyl}piperidine;
1-(3-{[4′-(piperidinomethyl)biphenyl-4-yl]oxy}propyl)piperidine;
(3S,5S)-1-{3-[4-(trans-4-morpholinocyclohex-1-yl)phenoxy]propyl}-3,5-dimethylpiperidine;
(3S)-1-{3-[4-(trans-4-morpholinocyclohex-1-yl)phenoxy]propyl}-3-methylpiperidine;
1-{3-[4-(trans-4-morpholinocyclohex-1-yl)phenoxy]propyl}piperidine;
1-{3-[4-(cis-4-morpholinocyclohex-1-yl)phenoxy]propyl}piperidine, dihydrochloride;
1-{3-[4-(trans-4-morpholinocyclohex-1-yl)phenoxy]propyl}piperidine, dihydrochloride;
(3S)-3-methyl-1-{3-[4-(cis-4-morpholinocyclohex-1-yl)phenoxy]propyl}piperidine, dihydrochloride;
(3S)-3-methyl-1-{3-[4-(trans-4-morpholinocyclohex-1-yl)phenoxy]propyl}piperidine, dihydrochloride;
1-(3-{[4′-(piperidinomethyl)biphenyl-4-yl]oxy}propyl)piperidine;
1-{3-[4-(4-piperidinobut-1-yn-1-yl)phenoxy]propyl}piperidine;
(E)-1-(3-{[4′-(3-piperidinoprop-1-en-1-yl)biphenyl-4-yl]oxy}propyl)piperidine;
(Z)-1-(3-{[4′-(3-piperidinoprop-1-en-1-yl)biphenyl-4-yl]oxy}propyl)piperidine;
1-methyl-4-[4′-(3-piperidinopropoxy)biphenyl]piperazine;
1-{3-[4-(cis-4-dimethylaminocyclohex-1-yl)methylphenoxy]propyl}piperidine;
1-{3-[4-(trans-4-dimethylaminocyclohex-1-yl)methylphenoxy]propyl}piperidine;
4-(3-{[4′-(3-piperidinopropyl)biphenyl-4-yl]oxy}propyl)piperidine;
(3S,5S)-1-{3-[4-(trans-4-aminocyclohex-1-yl)phenoxy]propyl}-3,5-dimethylpiperidine;
(3S)-4-{4-[3-(3-methylpiperidin-1-yl)propoxy]phenyl}pyridine 1-oxide;
(3S)-4-{4-[3-(3-methylpiperidin-1-yl)propoxy]phenyl}pyridine 1-oxide;
4-[4-(3-piperidinopropoxy)phenyl]pyridine 1-oxide;
2-methyl-4-(4-{3-[(3S)-3-methylpiperidin-1-yl]propoxy}phenyl)pyridine 1-oxide;
2-hydroxy-4-(4-{3-[(3S)-3-methylpiperidin-1-yl]propoxy}phenyl)pyridine;
1-methyl-4-(4-{3-[(3S)-3-methylpiperidin-1-yl]propoxy}phenyl)pyridinium;
2-(3-piperidinopropoxy)-4-(4-{3-[(3S)-3-methylpiperidin-1-yl]propoxy}phenyl)pyridine;
2-methyl-4-(4-{3-[(3S)-3-methylpiperidin-1-yl]propoxy}phenyl)pyridine;
1-{3-[4-(4-hydroxycyclohexyl)phenoxy]propyl}piperidine;
(3S)-1-{3-[trans-4-(4-hydroxycyclohexyl)phenoxy]propyl}-3-methylpiperidine;
(3S)-1-{3-[4-(4-hydroxycyclohexyl)phenoxy]propyl}-3-methylpiperidine;
1-{3-[trans-4-(4-hydroxycyclohexyl)phenoxy]propyl}pyrrolidine;
(3S)-1-{3-[4-(4-hydroxy-4-methylcyclohexyl)phenoxy]propyl}-3-methylpiperidine;
1-{3-[trans-4-(4-hydroxycyclohexyl)phenoxy]propyl}piperidine;
1-{3-[trans-4-(4-hydroxycyclohexyl)phenoxy]propyl}-2-methylpyrrolidine;
1-methyl-4-[4-(3-piperidinopropoxy)benzyloxy]piperidine;
1-methyl-4-[4-(3-piperidinopropoxy)benzyloxymethyl]piperidine;
1-methyl-4-{2-[4-(3-piperidinopropoxy)benzyloxy]ethyl}piperidine;
1-ethyl-3-[4-(3-piperidinopropoxy)benzyloxy]piperidine.

Preferably said antagonist or inverse agonist is not a compound described in patent application US 2005/0222151 filed by Johnson&Johnson nor a compound described in patent application WO2006/138714 filed by Janssen.

More specifically, said antagonist or inverse agonist is preferably not a compound represented by formula (V):

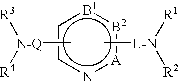

formula V where
in the rings containing A and B:
1) A, B¹ and B² are CH
2) A is CH, one of B¹ and B² is N, the other of B¹ and B² is CH; or
3) A is absent, B¹ is CH, and B² is O;
L is C₁₋₄ alkylene or a covalent bond;
Q is —(CH₂)—O—, —(CH₂)nC≡C— (where the moities —O— and —C≡C— are attached to the ring), carbonyl, or thiocarbonyl;
m is 2, 3 or 4
n is 1, 2, 3 or 4
R¹ optionally mono or di-substituted with R^{p}, is independently selected in the group consisting of H, C₁₋₇ alkyl, C₂₋₇ alkynyl, C₃₋₇ cycloalkyl, phenyl, benzyl, pyridinyl, pyrimidinyl, furanyl, thienyl, pyrrolyl, and an aromatic heterocycle with 5, 6 or 7 elements having 1 or 2 heteroatoms selected from among O, S, —N═, >NH, and >NC₁₋₄alkyl having 0, 1, or 2 double bonds;
R², optionally mono or di-substituted with R^{p}, is independently selected in the group consisting of C₁₋₇ alkyl, C₂₋₇ alkynyl, C₃₋₇ cycloalkyl, phenyl, benzyl, pyridinyl, pyrimidinyl, furanyl, thienyl, pyrrolyl, and a non-aromatic heterocycle with 5, 6 or 7 elements having 1 or 2 heteroatoms selected from among O, S, —N═, >NH, and >NC₁₋₄alkyl, having 0, 1, or 2 double bonds;
   or, alternatively
R¹ and R² can both be bound to the connecting nitrogen to form a ring, said ring being selected in the group consisting of:
1) a non-aromatic heterocycle with 4 to 7 elements, said heterocycle having 0 or 1 additional heteratom separated from the connecting nitrogen by at least one carbon and selected from among O, S, —N═, >NH, and >NC₁₋₄ alkyl, having 0, 1, or 2 double bonds having 0, 1, or 2 carbon elements which is a carbonyl having 0, 1 or 2 substituents R^{q}
2) a non-aromatic heterocycle with 4 to 7 elements, fused to benzo or pyrido group, said heterocyclic ring having 0 or 1 additional heteroatom separated from the connecting nitrogen by at least one carbon and selected from among O, S, —N═, >NH and >NC₁₋₄alkyl, having 0 or 1 additional double bond, having 0, 1, 2 carbon elements which is a carbonyl and having 0, 1 or 2 substituents R^{q}.
R^{p} is independently selected in the group consisting of —C₁₋₆alkyl, C₂₋₆alkenyl, C₃₋₆cycloalkyl, phenyl, pyridyl, furanyl, thienyl, benzyl, pyrimidinyl, pyrrolyl, halo, —OH, —OC₁₋₆alkyl, —OC₃₋₆cycloalkyl, —O-phenyl, —O-benzyl, —SH, —SC₁₋₆alkyl, SC₃₋₆cycloalkyl, —S-phenyl, —S-benzyl, —CN, —NO₂, —N(R^{y})R^{z} (where R^{y} and R^{z} are independently selected from among H and C₁₋₄ alkyl; or R^{y} and R^{z} can both be bound to the connecting nitrogen to form a monocyclic heterocycle with 5, 6 or 7 elements, selected from among O, S, —N═, >NH, and >NC₁₋₄alkyl, said ring optionally being substituted with —C₁₋₄alkyl, —OH, —OC₁₋₄alkyl, halo, or —COOC₁₋₄alkyl), —(C═O)N(R^{y})R^{z}, —(C═O)C₁₋₄alkyl, —SCF₃, —OCF₃, —CF₃, and —COOC alkyl, and —COOH;
R^{q} is independently selected in the group consisting of —C₁₋₆alkyl, halo, —OH, —OC₁₋₆alkyl, CN, —NO₂, —CF₃, and —COOC₁₋₄alkyl,
R³ optionally mono- or di-substituted with R⁵, is independently selected in the group consisting of —H, —C₁₋₇alkyl, C₂₋₇alkenyl, —C₂₋₇alkynyl, —C₃₋₇cycloalkyl, phenyl, benzyl, pyridinyl, pyrimidinyl, furanyl, thienyl, pyrrolyl, and a non-aromatic monocyclic heterocycle with 5, 6 or 7 elements having one or two heteroatoms selected from among O, S, —N═, >NH, and >NC₁₋₄ alkyl, having 0, 1, or 2 double bonds; and
R⁴, optionally mono- or di-substituted with R^{s}, is independently selected in the group consisting of —C₁₋₇alkyl, C₂₋₇alkenyl, —C₂₋₇alkynyl, —C₃₋₇cycloalkyl, phenyl, benzyl, pyridinyl, pyrimidinyl, furanyl, thienyl, pyrrolyl, and a non-aromatic monocyclic heterocycle with 5, 6 or 7 elements having one or two heteroatoms selected from among O, S, —N═, >NH, and >NC₁₋₄ alkyl, having 0, 1, or 2 double bonds;
R^{s} is independently selected in the group consisting of —C₁₋₆alkyl, —C₂₋₆alkenyl, —C₃₋₆cycloalkyl, phenyl, pyridyl, furanyl, thienyl, benzyl, pyrimidinyl, pyrrolyl, halo, —OH, —OC₁₋₆alkyl, —OC₃₋₆cycloalkyl, —O-phenyl, —O-benzyl, —SH, —SC₁₋₆alkyl, —SC₃₋₆cycloalkyl, —S-phenyl, —S-benzyl, —CN, —NO2, —N(R^{y})R^{z} where R^{y} and R^{z} are independently selected from among H and C₁₋₄alkyl; or R^{y} and R^{z} can both be bound to the connecting nitrogen to form a monocyclic heterocycle with 5, 6 or 7 elements selected from among O, S, —N═, >NH, and >NC₁₋₄ alkyl, said ring optionally being substituted with —C₁₋₄ alkyl, —OH, —OC₁₋₄ alkyl, halo, or —COOC₁₋₄alkyl), —(C═O)N(R^{y})R^{z}, —(C═O)C₁₋₄ alkyl, —SCF₃, —OCF₃, —CF₃, and —COOC₁₋₄ alkyl, and —COOH;
   or alternatively
R³ and R⁴ can both be bound to the connecting nitrogen to form a ring, said ring being selected in the group consisting of:
1) a non-aromatic heterocycle with 4 to 7 elements, said heterocycle having 0 or 1 additional heteroatom separated from the connecting nitrogen by at least one carbon and selected from among O, S, —N═, >NH, and >NC₁₋₄alkyl, having 0, 1, or 2 double bonds having 0, 1, or 2 carbon elements which is a carbonyl having 0, 1 or 2 substituents R^{t} and
2) a non-aromatic heterocycle resulting from the fusion of a benzo or pyrido group to an aromatic ring with 4 to 7 elements, said heterocycle having 0 or 1 additional heteroatom separated from the connecting nitrogen by at least one carbon and selected from among O, S, —N═, >NH et >NC₁alkyl, having 0 or 1 additional double bond, having 0, 1, 2 carbon elements which is a carbonyl and having 0, 1, or 2 substituents R^{t},
R^{t} is independently selected in the group consisting of —C₁₋₆alkyl, halo, —OH, —OC₁₋₆alkyl, —CN, —NO₂, —CF₃, and —COOC₁₋₄alkyl;
   and the enantiomers, diastereomers, hydrates, solvates and pharmaceutically acceptable salts, esters and amides thereof,
   and said antagonist or inverse agonist is also not a compound represented by formula (W):

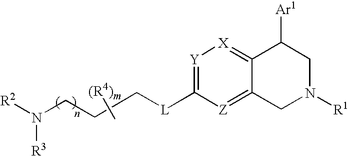

formula W where
One or two of X, Y and Z is N, and the rest of X, Y and Z is CR₅; L is —O— or —CH₂— and n is 1 or 2; L is —C≡C— and n is 0 or 1; m is 0, 1, or 2;
R1 is —H, or it is —C₁₋₆alkyl, —C₃₋₆alkenyl, —C₃₋₆alkynyl, —C₁₋₆alkylC₃₋₇cycloalkyl, —COOC₁₋₆alkyl, or —COObenzyl, each optionally mono-, di-, or tri-substituted with R^{a};
R^{a} is selected in the group consisting of —OH, —OC₁₋₆alkyl, phenyl optionally substituted with —OC₁₋₄alkyl or halo, —CN, —NO₂, —N(R^{b})R^{c} (where R^{b} and R^{c} are independently —H or —C₁₋₆alkyl), —C(O)N(R^{b})R^{c}, —N(R^{b})C(O)R^{b}, —N(Rb)SO₂C₁₋₆alkyl, —C(O)C₁₋₆alkyl, —S(O)₀₋₂—C₁₋₆alkyl, SO₂N(R^{b})R^{c}, —SCF₃, halo, —CF₃, —OCF₃, —COOH, and —COOC₁₋₆alkyl;
R² and R³ are independently selected from among —H, or in the group consisting of:
A) —C₁₋₆alkyl, —C₃₋₆alkenyl, —C₃₋₆alkynyl, C₃₋₇cycloalkylyl, —C₁₋₆alkylC₃₋₇cycloalkyl, benzyl;
B) phenyl or pyridyl, optionally fused by two adjacent carbons to a hydrocarbon with 3 or 4 elements to form an aromatic ring having 5 or 6 elements which has one carbon atom replaced by >O, >S, >NH or >N(C1-4alkyl) and which has at most one carbon atom optionally replaced by —N═;
C) a heterocycle with 4 to 8 elements, said heterocycle having one carbon atom which is the point of attachment, having 1 or 2 heteroatoms selected from among >O, >S(O)₀₋₂, and >NH, and having 0 or 1 double bond; and
D) an aromatic monocyclic hydrocarbon having 5 or 6 atoms in the ring, having one carbon atom which is the point of attachment, having one carbon atom replaced by >O, >S, >NH or >N(C1-4alkyl), having at most one additional carbon atom optionally replaced by —N═, and optionally fused with benzene or pyridine;
Where each of A) to D) is optionally mono-, di-, or tri-substituted with a group selected from among —OH, —C₁₋₄ alkylOH, —OC₁₋₆alkyl, —CN, —NO₂, —N(R^{d})R^{e} (where R^{d} and R^{e} are independently —H or C₁₋₆alkyl), —C(O)N(R^{d})R^{e}, —N(R^{d})C(O)R^{d}, —N(R^{d})SO₂C₁₋₆alkyl, —C(O)C₁₋₆alkyl, S(O)₀₋₂—C₁₋₆alkyl, SO₂N(R^{d})R^{e}, —SCF₃, halo, —CF₃, —OCF₃, —COOH, —COOC₁₋₆alkyl, —OC(O)N(R^{d})R^{e}, and —OC(O)OR^{d};
or, alternatively,
R² and R³ can both be bound to the nitrogen by which they are attached to form a heterocycle with 4 to 8 elements, said heterocycle having 0 or 1 additional heteroatom separated from the connecting nitrogen by at least one carbon and selected in the group consisting of >O, >(O)₀₋₂, >NH, and >NR^{f}, having 0 or 1 double bond, having 0, 1 or 2 carbons separated from the connecting nitrogen by at least one carbon which is a carbonyl, optionally fused with benzene or pyridine, optionally having one carbon which forms a bridge, and having 0 to 5 carbon substituents R^{ff},
R^{f} is selected in the group consisting of —C₁₋₆alkyl optionally mono-, di-, or tri-substituted with halo, —C₃₋₆alkenyl, —C₃₋₆alkynyl, —C₃₋₇ cycloalkyl, —C₁₋₆alkylC₃₋₇cycloalkyl, —C₂₋₆alkylOH, —C(O)N(R^{g})R^{h} (where R^{g} and R^{h} are independently —H or —C₁₋₆alkyl), —C(O)Rⁱ (where Rⁱ is —C₁₋₆alkyl, —C₃₋₈cycloalkyl, phenyl, or an aromatic heterocycle with 5 or 6 elements, each optionally mono-, di-, or tri substituted with —C₁₋₃alkyl, —OH, —OC₁₋₆alkyl, —CF₃, or halo), —S(O)₀₋₂—C₁₋₆alkyl, and —COOC₁₋₆alkyl;
R^{ff} is selected in the group consisting of —C₁₋₆alkyl optionally mono, di, or tri substituted with halo, —C₃₋₆alkenyl, —C₂₋₆alkynyl, —C₃₋₇cycloalkyl, —C₁₋₆alkylC₃₋₇cycloalkyl, halo, —OH, —C₁₋₆alkylOH, —OC₁₋₆alkyl, —OC₂₋₃alkylO—, —CN, —NO₂, —N(R^{g})R^{h} (where R^{g} and R^{h} are independently —H or —C₁₋₆alkyl), —C(O)NR(^{g})R^{h}, —N(R⁹)C(O)R⁹, —N(R^{g})SO₂C₁₋₆alkyl, —C(O)Rⁱ (where Rⁱ is —C₁₋₆alkyl, —C₃₋₈ cycloalkyl, phenyl, or an aromatic heterocycle with 5 or 6 elements, each optionally mono-, di-, or tri substituted with —C₁₋₃ alkyl, —OH, —OC₁₋₆alkyl, —CF₃, or halo), —S(O)₀₋₂—C₁₋₆alkyl, —SO₂N(R^{y})R^{z}, —SCF₃, —OCF₃, —COOH, and —COOC₁₋₆alkyl;
R⁴ is —OH, —OC₁₋₆alkyl, —CF₃, —C₁₋₆alkyl, or halo, two substituents R⁴ can be bound together to form a methylene or ethylene, or one of the R⁴ is bound with R² to form a methylene, ethylene or propylene; where each methylene, ethylene or propylene is optionally substituted with —OH, —OC₁₋₆alkyl, —SC₁₋₆alkyl, —CF₃, —C₁₋₆alkyl, amine or halo;
R⁵ is selected in the group consisting of —H, —C₁₋₆alkyl, —OH, —OC₁₋₆alkyl, —SC₁₋₆alkyl, and halo;
Ar¹ is an aryl or heteroaryl ring selected in the group consisting of:
a) phenyl, optionally mono-, di-, or tri-substituted with Rⁱ or di-substituted with fluorine, —(CH₂)₂₋₃NH—, —(CH₂)₁₋₂NH(CH₂)—, —(CH₂)₂₋₃N(C₁₋₄alkyl)-, or —(CH2)₁₋₂N(C₁₋₄alkyl)(CH₂)—;
Rⁱ is selected in the group consisting of:
   1) —OH, —C₁₋₆alkyl, —OC₁₋₆alkyl optionally mono-, di-, or tri-substituted with halo, —C₂₋₆alkenyl, —OC₃₋₆alkenyl, —C₂₋₆alkynyl, —OC₃₋₆alkynyl, —C₃₋₆cycloalkyl, —OC₃₋₆cycloalkyl, —CN, —NO₂, —N(R^{k})R^{l} (where R^{k} and R^{l} are independently —H or —C₁₋₆alkyl, or R^{m} and Rⁿ both bound to the nitrogen to which they are attached, form a heterocycle with 4 to 8 elements having 1 or 2 heteroatoms selected from among >O, >S(O)₀₋₂, >NH, and >NC₁₋₆alkyl, having 0 or 1 double bond, having 0 or 1 carbonyl groups), —SO₂N(R^{m})Rⁿ, —SCF₃, halo, —CF₃, —COOH, —COOC₁₋₆alkyl and —COOC₃₋₇ cycloalkyl;
   2) a) a saturated or partially saturated heterocycle with 4 to 8 elements, having 1 or 2 heteroatoms selected from among >O, >S(O)₀₋₂, >NH, and >NC₁₋₆alkyl, having 0 or 1 carbonyl; said ring optionally being mono-, di-, or tri-substituted with R^{p};
   R^{p} is a substituent independently selected in the group consisting of —OH, —C₁₋₆alkyl, —OC₁₋₆alkyl, phenyl, —CN, —NO₂, —N(R^{q})R^{r} (where R^{q} and R^{r} are independently —H, —C₁₋₆alkyl, or —C₂₋₆alkenyl), —C(O)N(R^{q})R^{r}, —N(R^{q})C(O)R^{r}, —N(R^{q})SO2C₁₋₆alkyl, —C(O)C₁₋₆alkyl, —S(O)₀₋₂—C₁₋₆alkyl, —SO₂N(R^{q})R^{r}, —SCF₃, halo, —CF₃, —OCF₃, —OCHF₂, —COOH, and —COOC₁₋₆alkyl;
b) phenyl or pyridyl fused by two adjacent cyclic carbons to a hydrocarbon with 3 elements to form a fused aromatic ring with 5 elements, in which one carbon atom of the hydrocarbon is replaced by >O, >S, >NH, or >N(C₁₋₄alkyl), and in which one additional carbon atom in said hydrocarbon is optionally replaced by —N═, the fused rings optionally being mono-, di-, or tri-substituted with R^{t};
R^{t} is a substituent independently selected in the group consisting of —OH, —C₁₋₆alkyl, —OC₁₋₆alkyl, phenyl, —CN, —NO₂, —N(R^{u})R^{v} (where R^{u} and R^{v} are independently —H or —C₁₋₆alkyl), —C(O)N(R^{u})R^{v}, —N(R^{u})C(O)R^{v}, —N(R^{u})SO₂C₁₋₆alkyl, —C(O)C₁₋₆alkyl, S(O)₀₋₂—C₁₋₆alkyl, —SO₂N(R^{u})R^{v}, —SCF₃, halo, —CF₃, —OCF₃, OCHF₂, —COOH, and —COOC₁₋₆alkyl;
c) phenyl fused by two adjacent elements to a hydrocarbon with 4 elements to form a fused aromatic ring with 6 elements, in which 0, 1 or 2 carbon atoms are replaced by —N═, the fused rings optionally being mono-, di-, or tri-substituted with R^{t};
d) an aromatic monocyclic hydrocarbon with 5 elements, having one carbon atom which is the point of attachment, having one carbon atom replaced by >O, >S, >NH or >N(C₁₋₄alkyl), having at most one additional carbon atom optionally replaced by —N═, optionally mono- or di-substituted with R^{t}, and optionally fused with benzene or pyridine by two adjacent carbon atoms, the part fused with benzene or pyridine optionally being mono-, di-, or tri-substituted with R^{t}; and
e) an aromatic monocyclic hydrocarbon with 6 elements, having one carbon atom which is the point of attachment, having 1 or 2 carbon atoms replaced by —N═, optionally mono- or di-substituted with R^{t}, and optionally fused with benzene or pyridine by two adjacent carbon atoms, where the part fused with benzene or pyridine is optionally mono- or di-substituted with R^{t};
and the enantiomers, diastereomers, hydrates, solvates thereof and the pharmaceutically acceptable salts, esters and amides thereof.

### Pharmaceutical Compositions

As provided for in the invention, modafinil is intended to be administered as adjunct to a treatment with a histamine H3 receptor antagonist or inverse agonist so as to potentiate the therapeutic effects of said treatment on narcolepsy-cataplexy, in particular so as to potentiate the anticataplectic effect of the H3 receptor antagonist or inverse agonist.

Modafinil and the H3 receptor antagonist or inverse agonist may be present within a same pharmaceutical composition.

Alternatively, modafinil and the H3 receptor antagonist or inverse agonist are intended for separate administration, namely, either concomitantly, or independently, for example at different times.

The medicament according to the invention may be administered by any suitable route of administration, for example by the oral, rectal route or by the parenteral route, for example by intravenous, intracutaneous or intradermal injection. Preferably, oral administration is considered.

Therefore, the medicament may be formulated as tablets, capsules, powder or any solid oral form or any liquid oral preparation. The pharmaceutical composition generally comprises a physiologically acceptable medium, for example for preparing tablets or capsules or for a liquid preparation, such as the vehicles used in an altogether classic manner.

Whatever the route of administration and the form of the pharmaceutical composition, modafinil and the H3 receptor antagonist or inverse agonist are preferably administered in synergistic amounts with respect to the anticataplectic effect.

In the prior art, modafinil is usually administered at oral doses of 200 to 800 mg. The invention proposes combining doses of 5 to 50 mg, preferably 10 to 40 mg, of an H3 receptor antagonist or inverse agonist such as 1-{3-[3-(4-chlorophenyl)propoxy]propyl}piperidine, and doses of 50 to 500 mg, preferably 100 to 300 mg, more preferably only 100 to 150 mg of modafinil, allowing to reduce the side effects of the latter.

The invention therefore has as object a pharmaceutical composition comprising from 50 to 500 mg of modafinil, and 5 to 50 mg of an H3 receptor antagonist or inverse agonist.

The combination of modafinil and a histamine H3 receptor antagonist or inverse agonist is useful for treating narcolepsy-cataplexy and, more generally, is useful for treating disorders for which modafinil is indicated. Target diseases include disorders of sleep and wakefulness such as hypersomnia, narcolepsy (including narcolepsy-cataplexy), sleep apnea or hypopnea, fatigue, and shift work sleep disorders. Other targets include disorders of sleep and wakefulness associated with pathologies such as Parkinson's disease (Ondo et al., J. Neurol. Neurosurg. Psych. 2005, 76, 1636; Happe, J. Clin. Psychol., 2001, 57, 1559), Alzheimer's disease, multiple sclerosis, and the like. Attention disorders such as ADHD (attention deficit hyperactivity disorder) are also considered (Biederman et al., Pediatrics, 2005, 116, 777). Lastly, modafinil has been proposed for the treatment of depression, urinary incontinence and ischemia, pathologies for which combination with an H3 receptor antagonist or inverse agonist may also be useful, particularly in the treatment of sleep or wakefulness disorders associated with depression.

The invention also provides a method for treating said disorders, in particular narcolepsy-cataplexy, in a patient requiring such treatment, said method comprising administering to said patient a therapeutically effective amount of modafinil and of at least one H3 receptor antagonist or inverse agonist.

In the spirit of the invention, the term “treatment” should be understood to mean curative or preventative treatment of narcolepsy-cataplexy, and includes improvement in the symptoms of the disease, particularly the prevention, or reduction in the number of, cataplexy attacks.

Preferably, the patient is a human being, but may also be a non-human mammal.

### EXAMPLES

### Example 1

### Narcolepsy/Cataplexy Model

Continuous electroencephalographic (EEG) and electromyographic (EMG) recordings were obtained in groups of 6 orexin knockout mice (Chemilli et al., Cell, 1999, 98, 437).

The mice were given an intraperitoneal injection of either modafinil alone at a dose of 64 g/kg, or the compound 1-{3-[3-(4-chlorophenyl)propoxy]propyl}piperidine, a potent H3 receptor inverse agonist, at a dose of 20 mg/kg, or a combination of these two compounds, or else a vehicle (0.9% NaCl).

Analysis of the EEG and EMG data showed that:

1. In comparison with non-mutant mice, the null mice receiving vehicle alone showed decreased wakefulness, more episodes of REM sleep and direct transitions from wakefulness to REM sleep, an anomaly considered in this murine model to be the equivalent of narcolepsy-cataplexy attacks in humans (Willie et al., Neurosci. 2005, 130, 583);
2. 1-{3-[3-(4-chlorophenyl)propoxy]propyl}piperidine and modafinil produced a marked increase in wakefulness in the mutant mice;
3. While modafinil had no effect on wake-REM sleep transitions (in agreement with Willie et al., 2005, supra, and its lack of anticataplectic effect in human narcoleptics), this parameter was improved by 1-{3-[3-(4-chlorophenyl)propoxy]propyl}piperidine;
4. The combination of the two compounds led to an almost total disappearance of all symptoms in these mice, including cataplectic transitions and the alterations of rapid EEG rhythms associated with cognitive processes and which are abnormal in these animals.

This remarkable synergy, which was unforeseeable in so far as the mechanism of action of modafinil is basically unknown, is not a result of a metabolic interaction between the two compounds because they were administered at maximum dose. The combination of the two compounds provides a complete treatment of narcolepsy-cataplexy symptomatology, and allows a reduction in the usual modafinil doses.

### Example 2

### Parkinson's Disease Model

The inventors also investigated the combination of 1-{3-[3-(4-chlorophenyl)propoxy]propyl}piperidine and modafinil on wakefulness in cats with MPTP-induced parkinsonism. MPTP is a neurotoxin which selectively destroys dopaminergic neurons in the mesencephalon. MPTP-treated animals display sleepiness similar to that seen in many patients. Modafinil alone produced only a modest improvement in wakefulness in these animals (as in Parkinson patients), but when it was administered in combination with 1-{3-[3-(4-chlorophenyl)propoxy]propyl}piperidine, there was an almost complete normalization of this parameter.

1. Pharmaceutical composition comprising, in a pharmaceutically acceptable medium, modafinil and at least one histamine H3 receptor antagonist or inverse agonist, it being understood that said antagonist or inverse agonist is not a compound represented by formula (V): 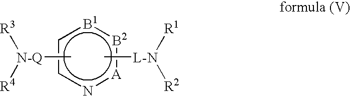
where in the rings containing A and B:
1) A, B¹ and B² are CH
2) A is CH, one of B¹ and B² is N, the other one of B¹ and B² is CH; or
3) A is absent, B¹ is CH, and B² is O;
L is C₁₋₄ alkylene or a covalent bond;
Q is —(CH₂)—O—, —(CH₂)nC≡C— (where the groups —O— and —C≡C— are attached to the ring), carbonyl, or thiocarbonyl;
m is 2, 3 or 4
n is 1, 2, 3, or 4
R¹, optionally mono- or di-substituted with R^{p}, is independently selected in the group consisting of H, C₁₋₇ alkyl, C₂₋₇ alkynyl, C₃₋₇ cycloalkyl, phenyl, benzyl, pyridinyl, pyrimidinyl, furanyl, thienyl, pyrrolyl, and an aromatic heterocycle with 5, 6, or 7 elements having 1 or 2 heteroatoms selected from among O, S, —N═, >NH, and >NC₁₋₄alkyl having 0, 1, or 2 double bonds;
R², optionally mono- or di-substituted with R^{p}, is independently selected in the group consisting of C₁₋₇ alkyl, C₂₋₇ alkynyl, C₃₋₇ cycloalkyl, phenyl, benzyl, pyridinyl, pyrimidinyl, furanyl, thienyl, pyrrolyl, and a non-aromatic heterocycle with 5, 6, or 7 elements having 1 or 2 heteroatoms selected from among O, S, —N═, >NH, and >NC₁₋₄alkyl, having 0, 1, or 2 double bonds;
or, alternatively
R¹ and R² can both be bound to the connecting nitrogen to form a ring, said ring being selected in the group consisting of:
1) a non-aromatic heterocycle with 4 to 7 elements, said heterocycle having 0 or 1 additional heteratom separated from the connecting nitrogen by at least one carbon and selected from among O, S, —N═, >NH, and >NC₁₋₄ alkyl, having 0, 1, or 2 double bonds having 0, 1, or 2 carbon elements which is a carbonyl having 0, 1 or 2 substituents R^{q} and
2) a non-aromatic heterocycle with 4 to 7 elements fused to benzo or pyrido, said heterocyclic ring having 0 or 1 additional heteroatom separated from the connecting nitrogen by at least one carbon and selected from among O, S, —N═, >NHC₁₋₄alkyl, having 0 or 1 additional double bond, having 0, 1 or 2 carbon elements which is a carbonyl and having 0, 1 or 2 substitutents R^{q}
R^{p} is independently selected in the group consisting of —C₁₋₆alkyl, C₂₋₆alkenyl, C₃₋₆cycloalkyl, phenyl, pyridyl, furanyl, thienyl, benzyl, pyrimidinyl, pyrrolyl, halo, —OH, —OC₁₋₆alkyl, —OC₃₋₆cycloalkyl, —O-phenyl, —O-benzyl, —SH, —SC₁₋₆alkyl, SC₃₋₆cycloalkyl, —S-phenyl, —S-benzyl, —CN, —NO₂, —N(R^{y})R^{z} (where R^{y} and R^{z} are independently selected from among H and C₁₋₄ alkyl; or R^{y} and R^{z} can both be bound to the connecting nitrogen to form a monocyclic heterocycle with 5, 6 or 7 elements selected from among O, S, —N═, >NH, and >NC₁₋₄alkyl, said ring optionally being substituted with —C₁₋₄alkyl, —OH, —OC₁₋₄alkyl, halo, or —COOC₁₋₄alkyl), —(C═O)N(R^{y})R^{z}, —(C═O)C₁₋₄alkyl, —SCF₃, —OCF₃, —CF₃, and —COOC alkyl, and —COOH;
R^{q} is independently selected in the group consisting of —C₁₋₆alkyl, halo, —OH, —OC₁₋₆alkyl, CN, —NO₂, —CF₃, and —COOC₁₋₄alkyl,
R³, optionally mono- or di-substituted with R⁵, is independently selected in the group consisting of —H, —C₁₋₇alkyl, C₂₋₇alkenyl, —C₂₋₇alkynyl, —C₃₋₇cycloalkyl, phenyl, benzyl, pyridinyl, pyrimidinyl, furanyl, thienyl, pyrrolyl, and a non-aromatic monocyclic heterocycle with 5, 6, or 7 elements having 1 or 2 heteroatoms selected from among O, S, —N═, >NH, and >NC₁₋₄ alkyl, having 0, 1, or 2 double bonds; and
R⁴, optionally mono- or di-substituted with R⁵, is independently selected in the group consisting of —C₁₋₇alkyl, C₂₋₇alkenyl, —C₂₋₇alkynyl, —C₃₋₇cycloalkyl, phenyl, benzyl, pyridinyl, pyrimidinyl, furanyl, thienyl, pyrrolyl, and a non-aromatic monocyclic heterocycle with 5, 6, or 7 elements having 1 or 2 heteroatoms selected from among O, S, —N═, >NH, and >NC₁₋₄ alkyl, having 0, 1, or 2 double bonds;
R^{s} is independently selected in the group consisting of —C₁₋₆alkyl, —C₂₋₆alkenyl, —C₃₋₆cycloalkyl, phenyl, pyridyl, furanyl, thienyl, benzyl, pyrimidinyl, pyrrolyl, halo, —OH, —OC₁₋₆alkyl, —OC₃₋₆cycloalkyl, —O-phenyl, —O-benzyl, —SH, —SC₁₋₆alkyl, —SC₃₋₆cycloalkyl, —S-phenyl, —S-benzyl, —CN, —NO2, —N(R^{y})R^{z} where R^{y} and R^{z} are independently selected from among H and C₁₋₄ alkyl; or R^{y} and R^{z} can both be bound to the connecting nitrogen to form a monocyclic heterocycle with 5, 6, or 7 elements selected from among O, S, —N═, >NH, and >NC₁₋₄ alkyl, said ring optionally being substituted with —C₁₋₄ alkyl, —OH, —OC₁₋₄ alkyl, halo, or —COOC₁₋₄alkyl), —(C═O)N(R^{y})R^{z}, —(C═O)C₁₋₄ alkyl, —SCF₃, —OCF₃, —CF₃, and —COOC₁₋₄ alkyl, and —COOH;
or, alternatively
R³ and R⁴ can both be bound to the connecting nitrogen to form a ring, said ring is selected in the group consisting of:
1) a non-aromatic heterocycle with 4 to 7 elements, said heterocycle having 0 or 1 additional heteroatom separated from the connecting nitrogen by at least one carbon and selected from among O, S, —N═, >NH, and >NC₁₋₄ alkyl, having 0, 1, or 2 double bonds having 0, 1, or 2 carbon elements which is a carbonyl having 0, 1 or 2 substituents R^{t} and
2) a non-aromatic heterocycle resulting from the fusion of a benzo or pyrido to an aromatic ring with 4 to 7 elements, said heterocycle having 0 or 1 additional heteroatom separated from the connecting nitrogen by at least one carbon and selected from among O, S, —N═, >NH and >NClalkyl, having 0 or 1 additional double bond, having 0, 1, 2 carbon elements which is a carbonyl and having 0, 1, or 2 substituents R^{t},
R^{t} is independently selected in the group consisting of —C₁₋₆alkyl, halo, —OH, —OC₁₋₆alkyl, —CN, —NO₂, —CF₃, and —COOC₁₋₄alkyl;
and the enantiomers, diastereomers, hydrates, solvates and pharmaceutically acceptable salts, esters and amides thereof,
said antagonist or inverse agonist also not corresponding to a compound represented by formula (W): 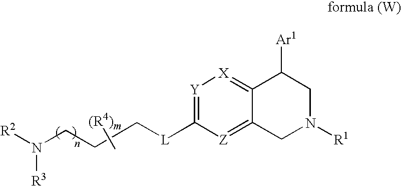
where one or two of X, Y and Z is N, and the rest of X, Y and Z is CR₅; L is —O— or —CH₂— and n is 1 or 2; L is —C≡C— and n is 0 or 1; m is 0, 1, or 2;
R1 is —H, or is —C₁₋₆alkyl, —C₃₋₆alkenyl, —C₃₋₆alkynyl, —C₁₋₆alkylC₃₋₇cycloalkyl, —COOC₁₋₆alkyl, or —COObenzyl, each optionally being mono-, di-, tri-substituted with R^{a};
R^{a} is selected in the group consisting of —OH, —OC₁₋₆alkyl, phenyl optionally substituted by —OC₁₋₄ alkyl or halo, —CN, —NO₂, —N(R^{b})R^{c} (where R^{b} and R^{c} are independently —H or —C₁₋₆alkyl), —C(O)N(R^{b})R^{c}, —N(R^{b})C(O)R^{b},
—N(Rb)SO₂C₁₋₆alkyl, —C(O)C₁₋₆alkyl, —S(O)₀₋₂—C₁₋₆alkyl, SO₂N(R^{b})R^{c}, —SCF₃, halo, —CF₃, —OCF₃, —COOH, and —COOC₁₋₆alkyl;
R and R³ are independently selected from among —H, or in the group consisting of:
A) —C₁₋₆alkyl, —C₃₋₆alkenyl, —C₃₋₆alkynyl, C₃₋₇cycloalkylyl, —C₁₋₆alkylC₃₋₇cycloalkyl, benzyl;
B) phenyl or pyridyl, optionally fused by two adjacent carbons to a hydrocarbon with 3 or 4 elements to form an aromatic ring with 5 or 6 elements which has one carbon atom replaced by >O, >S, >NH or >N(C₁₋₄alkyl) and which has at most one carbon atom optionally replaced by —N═;
C) a heterocycle with 4 to 8 elements, said heterocycle having one carbon atom which is the point of attachment, having 1 or 2 heteroatoms selected from among >O, >S(O)₀₋₂, and >NH, and having 0 or 1 double bond; and
D) an aromatic monocyclic hydrocarbon having 5 or 6 atoms in the ring, having one carbon atom which is the point of attachment, having one carbon atom replaced by >O, >S, >NH, or >N(C₁₋₄ alkyl), having at most one additional carbon atom optionally replaced by —N═, and optionally fused to benzene or pyridine;
Where each of A) to D) is optionally mono-, di-, or tri-substituted with a group selected in the group consisting of —OH, —C₁₋₄ alkylOH, —OC₁₋₆alkyl, —CN, —NO₂, —N(R^{d})R^{e} (where R^{d} and R^{e} are independently —H or C₁₋₆alkyl), —C(O)N(R^{d})R^{e}, —N(R^{d})C(O)R^{d}, —N(R^{d})SO₂C₁₋₆alkyl, —C(O)C₁₋₆alkyl, S(O)₀₋₂—C₁₋₆alkyl, SO₂N(R^{d})R^{e}, —SCF₃, halo, —CF₃, —OCF₃, —COOH, —COOC₁₋₆alkyl, —OC(O)N(R^{d})R^{e}, and —OC(O)OR^{d};
or, alternatively,
R² and R³ can both be bound to the nitrogen by which they are attached to form a heterocycle with 4 to 8 elements, said heterocycle having 0 or 1 additional heteroatom separated from the connecting nitrogen by at least one carbon and selected from among >O, >(O)₀₋₂, >NH, and >NR^{f}, having 0 or 1 double bond, having 0, 1 or 2 carbons separated from the connecting nitrogen by at least one carbon which is a carbonyl, optionally fused with benzene or pyridine, optionally having a carbon which forms a bridge, and having 0 to 5 carbon substituents R^{ff},
R^{f} is selected in the group consisting of —C₁₋₆alkyl optionally mono-, di-, or tri-substituted with halo, —C₃₋₆alkenyl, —C₃₋₆alkynyl, —C₃₋₇ cycloalkyl, —C₁₋₆alkylC₃₋₇cycloalkyl, —C₂₋₆alkylOH, —C(O)N(R^{g})R^{h} (where R^{g} and R^{h} are independently —H or —C₁₋₆alkyl), —C(O)Rⁱ (where Rⁱ is —C₁₋₆alkyl, —C₃₋₈cycloalkyl, phenyl, or an aromatic heterocycle with 5 or 6 elements, each optionally mono-, di-, or tri-substituted with —C₁₋₃alkyl, —OH, —OC₁₋₆alkyl, —CF₃, or halo), —S(O)₀₋₂—C₁₋₆alkyl, and —COOC₁₋₆alkyl;
R^{ff} is selected in the group consisting of —C₁₋₆alkyl optionally mono-, di-, or tri-substituted with halo, —C₃₋₆alkenyl, —C₂₋₆alkynyl, —C₃₋₇cycloalkyl, —C₁₋₆alkylC₃₋₇cycloalkyl, halo, —OH, —C₁₋₆alkylOH, —OC₁₋₆alkyl, —OC₂₋₃alkylO—, —CN, —NO₂, —N(R^{g})R^{h} (where R^{g} and R^{h} are independently —H or —C₁₋₆alkyl), —C(O)NR(^{g})R^{h}, —N(R^{g})C(O)R^{g}, —N(R^{g})SO₂C₁₋₆alkyl, —C(O)Rⁱ (where Rⁱ is —C₁₋₆alkyl, —C₃₋₈ cycloalkyl, phenyl, or an aromatic heterocycle with 5 or 6 elements, each optionally mono-, di-, or tri substituted with —C₁₋₃ alkyl, —OH, —OC₁₋₆alkyl, —CF₃, or halo), —S(O)₀₋₂—C₁₋₆alkyl, —SO₂N(R^{y})R^{z}, —SCF₃, —OCF₃, —COOH, and —COOC₁₋₆alkyl;
R⁴ is —OH, —OC₁₋₆alkyl, —CF₃, —C₁₋₆alkyl, or halo, two substituents R⁴ can be bound together to form a methylene or an ethylene, or one of the R⁴ is bound to R² to form a methylene, ethylene or propylene; in which each methylene, ethylene or propylene is optionally substituted with —OH, —OC₁₋₆alkyl, —SC₁₋₆alkyl, —CF₃, —C₁₋₆alkyl, amine or halo;
R⁵ is selected in the group consisting of —H, —C₁₋₆alkyl, —OH, —OC₁₋₆alkyl, —SC₁₋₆alkyl, and halo;
Ar¹ is an aryl or heteroaryl ring selected in the group consisting of:
a) phenyl, optionally mono-, di-, or tri-substituted with Rⁱ or di-substituted with fluorine, —(CH₂)₂₋₃NH—, —(CH₂)₁₋₂NH(CH₂)—, —(CH₂)₂₋₃N(C₁₋₄alkyl)-, or
b) —(CH₂)₁₋₂N(C₁₋₄alkyl)(CH₂)—;
Rⁱ is selected in the group consisting of:
1) —OH, —C₁₋₆alkyl, —OC₁₋₆alkyl optionally mono-, di-, or tri-substituted with halo, —C₂₋₆alkenyl, —OC₃₋₆alkenyl, —C₂₋₆alkynyl, —OC₃₋₆alkynyl, —C₃₋₆cycloalkyl, —OC₃₋₆cycloalkyl, —CN, —NO₂, —N(R^{k})R^{l} (where R^{k} and R^{l} are independently —H or —C₁₋₆alkyl, or R^{m} and Rⁿ bound together with their connecting nitrogen form a heterocycle with 4 to 8 elements having 1 or 2 heteroatoms selected from among >O, >S(O)₀₋₂, >NH, and >NC₁₋₆alkyl, having 0 or 1 double bond, having 0 or 1 carbonyl groups), —SO₂N(R^{m})Rⁿ, —SCF₃, halo, —CF₃, —COOH, —COOC₁₋₆alkyl and —COOC₃₋₇cycloalkyl; and
2) a) a saturated or partially saturated heterocycle with 4 to 8 elements, having 1 or 2 heteroatoms selected from among >O, >S(O)₀₋₂, >NH, and >NC₁₋₆alkyl, having 0 or 1 carbonyl; said ring optionally being mono-, di-, or tri-substituted with R^{p};
R^{P} is a substituent independently selected in the group consisting of —OH, —C₁₋₆alkyl, —OC₁₋₆alkyl, phenyl, —CN, —NO₂, —N(R^{q})R^{r} (where R^{q} and R^{r} are independently —H, —C₁₋₆alkyl, or —C₂₋₆alkenyl), —C(O)N(R^{q})R^{r}, —N(R^{q})C(O)R^{r}, —N(R^{q})SO2C₁₋₆alkyl, —C(O)C₁₋₆alkyl, —S(O)0₋₂—C₁₋₆alkyl, —SO₂N(R^{q})R^{r}, —SCF₃, halo, —CF₃, —OCF₃, —OCHF₂, —COOH, and —COOC₁₋₆alkyl;
c) phenyl or pyridyl fused by two adjacent cyclic carbon atoms to a hydrocarbon with 3 elements to form a fused aromatic ring with 5 elements, where one carbon atom of the hydrocarbon is replaced by >O, >S, >NH, or >N(C₁₋₄alkyl), and where one additional carbon atom in said hydrocarbon is replaced by —N═, the fused rings optionally being mono-, di-, or tri-substituted with R^{t};
R^{t} is a substituent independently selected in the group consisting of —OH, —C₁₋₆alkyl, —OC₁₋₆alkyl, phenyl, —CN, —NO₂, —N(R^{u})R^{v} (where R^{u} and R^{v} are independently —H or —C₁₋₆alkyl), —C(O)N(R^{u})R^{v}, —N(R^{u})C(O)R^{v}, —N(R^{u})SO₂C₁₋₆alkyl, —C(O)C₁₋₆alkyl, S(O)0₋₂—C₁₋₆alkyl, —SO₂N(R^{u})R^{v}, —SCF₃, halo, —CF₃, —OCF₃, OCHF₂, —COOH, and —COOC₁₋₆alkyl;
d) phenyl fused by two adjacent elements to a hydrocarbon with 4 elements to form a fused aromatic ring with 6 elements, where 0, 1 or 2 carbon atoms are replaced by —N═, the fused rings optionally being mono-, di-, or tri-substituted with R^{t};
e) an aromatic monocyclic hydrocarbon with 5 elements, having one carbon atom which is the point of attachment, having one carbon atom replaced by >O, >S, >NH, or >N(C₁₋₄ alkyl), having at most one additional carbon atom replaced by —N═, optionally mono- or di-substituted with R^{t}, and optionally fused with benzene or pyridine by two adjacent carbon atoms, the part fused with benzene or pyridine optionally being mono-, di- or tri-substituted with R^{t}; and
f) an aromatic monocyclic hydrocarbon with 6 elements, having one carbon atom which is the point of attachment, having 1 or 2 carbon atoms replaced by —N═, optionally mono- or di-substituted with R^{t}, and optionally fused with benzene or pyridine by two adjacent carbon atoms, where the part fused with benzene or pyridine is optionally mono- or di-substituted with R^{t};
and the enantiomers, diastereomers, hydrates, solvates thereof and the pharmaceutically acceptable salts, esters and amides thereof.

2. Composition according to claim 1, wherein the H3 receptor antagonist or inverse agonist is a compound represented by formula (I): 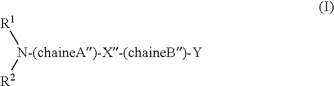
where:
—NR¹R² represents a piperidyl group unsubstituted or substituted with one or more alkyl groups, preferably methyl groups;
the A″ chain is a —(CH₂)ₓ— chain with x being a whole number from 1 to 6, preferably from 1 to 4, more preferably x=3;
X″ is an oxygen atom;
the B″ chain is a —(CH₂)_{y}— group with y being a whole number from 1 to 4, preferably y=2 or y=3;
Y″ is a phenyl group unsubstituted or substituted with one or more halogen atoms, or with one or more alkyl groups.

3. Composition according to claim 2, wherein the H3 receptor antagonist or inverse agonist is a compound represented by formula (I) where —NR¹R² represents an unsubstituted piperidyl group, and Y″ is a phenyl group substituted with a halogen atom, preferably chlorine.

4. Composition according to claim 3, wherein the antagonist or inverse agonist is 1-{3-[3-(4-chlorophenyl)propoxy]propyl}piperidine.

5. Composition according to claim 4, in a form suitable for oral administration.

6. Composition according to claim 5, comprising from 50 to 500 mg of modafinil, and from 5 to 50 mg of H3 receptor inverse agonist.

7. A method for treating a disorder of sleep, wakefulness or vigilance, which method comprises administering modafinil in combination with at least one histamine H3 receptor antagonist or inverse agonist, which is not a compound represented by formula V or by formula W such as defined in claim 1.

8. The method according to claim 7, wherein the medicament is a pharmaceutical composition comprising, in a physiologically acceptable medium, modafinil and at least one histamine H3 receptor antagonist or inverse agonist.

9. The method according to claim 8, in which modafinil and the H3 receptor antagonist or inverse agonist are intended for separate administration.

10. The method according to claim 7, wherein the H3 receptor antagonist or inverse agonist is a compound represented by formula (I): 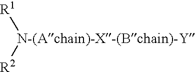
where:
—NR¹R² represents a piperidyl group unsubstituted or substituted with one or more alkyl groups, preferably methyl groups;
the A″ chain is a —(CH₂)ₓ— chain with x being a whole number from 1 to 6, preferably from 1 to 4, more preferably x=3;
X″ is an oxygen atom;
the B″ chain is a —(CH₂)_{y}— group with y being a whole number from 1 to 4, preferably y=2 or y=3;
Y″ is a phenyl group unsubstituted or substituted with one or more halogen atoms, or with one or more alkyl groups.

11. The method according to claim 10, wherein the H3 receptor antagonist or inverse agonist is a compound represented by formula (I) where —NR¹R² represents an unsubstituted piperidyl group, and Y″ is a phenyl group substituted with a halogen atom, preferably chlorine.

12. The method according to claim 11, wherein the antagonist or inverse agonist is 1-{3-[3-(4-chlorophenyl)propoxy]propyl}piperidine.

13. The method according to claim 7, wherein modafinil and the H3 receptor antagonist or inverse agonist are intended for oral administration.

14. The method according to claim 13, wherein modafinil is intended to be administered to a patient at a dose of 50 to 500 mg, and the H3 receptor antagonist or inverse agonist is intended to be administered to a patient at a dose of 5 to 50 mg of H3 receptor antagonist or inverse agonist.

15. The method according to claim 7, wherein the disorder of sleep or wakefulness is selected in the group consisting of hypersomnia, narcolepsy, sleep apnea or hypopnea, fatigue, shift work sleep disorders, disorders of sleep and wakefulness associated with Parkinson's disease, Alzheimer's disease or multiple sclerosis, or else ADHS (attention deficit hyperactivity disorder).

16. The method according to claim 15, wherein the narcolepsy is narcolepsy-cataplexy.

17. The method according to claim 16, for the prevention of cataplexy attacks.

18. Kit comprising, within the same package,
a pharmaceutical composition A comprising modafinil in a physiologically acceptable medium;
a pharmaceutical composition B comprising a histamine H3 receptor antagonist or inverse agonist, in a physiologically acceptable medium, it being understood that said antagonist or said inverse agonist is not a compound represented by formula V or by formula W such as defined in claim 1.

19. The Kit according to claim 18, wherein the H3 receptor antagonist or inverse agonist is 1-{3-[3-(4-chlorophenyl)propoxy]propyl}piperidine.
